(19) United States
(12) Patent Application Publication
(10) Pub. No.: US 2022/0280059 A1
(43) Pub. Date: Sep. 8, 2022

(54) METHOD OF BIOIMPEDANCE TECHNOLOGY TO EVALUATE LOCAL OR WHOLE BODY BONE MINERAL DENSITY

(71) Applicant: STARBIA MEDITEK CO.,LTD., TAICHUNG CITY (TW)

(72) Inventor: Kuen-Chang HSIEH, TAICHUNG CITY (TW)

(21) Appl. No.: 17/687,022

(22) Filed: Mar. 4, 2022

(30) Foreign Application Priority Data
Mar. 5, 2021 (TW) 110107862

(51) Int. Cl. A61B 5/0537; A61B 5/00; G16H 50/30

(52) U.S. Cl. CPC A61B 5/0537; A61B 5/4509; G16H 50/30

(57) ABSTRACT

A method of bioimpedance technology to evaluate local or whole body bone mineral density includes the steps of (a) measuring the resistance and reactance of a subject by a bioimpedance measuring instrument and obtaining the height of the subject, and (b) calculating the bone mineral density of the local or whole body of the subject by the formula: BMD=a+b R/H+d Xc/H, where a, b, and d are weighting coefficients, R is the resistance of the subject; Xc is the reactance of the subject; H is the height of the subject. This can achieve the advantages of reducing measurement costs and rapid measurement.

### BACKGROUND OF THE INVENTION

### 1. Field of the Invention

The present invention is related to bone mineral density evaluation technology, and particularly refers to a method of bioimpedance technology to evaluate local or whole body bone mineral density.

### 2. Description of the Related Art

Most of the current bone mass inspection methods are measured according to the different absorption levels of ionized radiation by bone and soft tissue. Among them, the use of dual energy X-ray absorptiometry (DXA) to express bone mineral density (BMD) by mineral mass (g/cm²) is widely accepted.

However, although the method of applying DXA to measuring BMD is accepted by everyone, because DXA measurement is expensive, and its measurement location must be implemented in a hospital or related professional institution, it cannot be applied to home health care.

In addition, each measurement of DXA takes tens of minutes. Therefore, the current method of measuring bone mineral density using DXA is costly and time-consuming, and it is not convenient to use.

### SUMMARY OF THE INVENTION

The present invention has been accomplished under the circumstances in view. It is the main object of the present invention to provide a method of bioimpedance technology to evaluate local or whole body bone mineral density, which, compared with the prior art, is low in cost, fast, and convenient in use.

In order to achieve the above-mentioned purpose, a method of bioimpedance technology to evaluate local or whole body bone mineral density provided by the present invention comprises the following steps:

Step (a) Measure the resistance (R) and reactance (Xc) of a subject with a bioimpedance measuring instrument, and obtain the height of the subject.

Step (b) The bone mineral density of the subject's local or whole body is calculated by the following calculation formula:

BMD=a+b R/H+d Xc/H, where a, b, and d are weighting coefficients; R is the resistance of the subject; Xc is the reactance of the subject; H is the height of the subject.

Thereby, a method of bioimpedance technology to evaluate local or whole body bone mineral density provided by the present invention only uses a bioimpedance measuring instrument to obtain the resistance, reactance, and height of the subject, and then calculates the formula BMD=a+b R/H+d Xc/H to obtain the bone mineral density. Compared with the prior art, the present invention is low in cost, fast, and convenient in use.

### BRIEF DESCRIPTION OF THE DRAWINGS

FIG. 1 is a flowchart of a preferred embodiment of the present invention.

FIG. 2 is a block diagram of a preferred embodiment of the present invention.

FIG. 3 is a GLM regression analysis diagram of a preferred embodiment of the present invention.

### DETAILED DESCRIPTION OF THE INVENTION

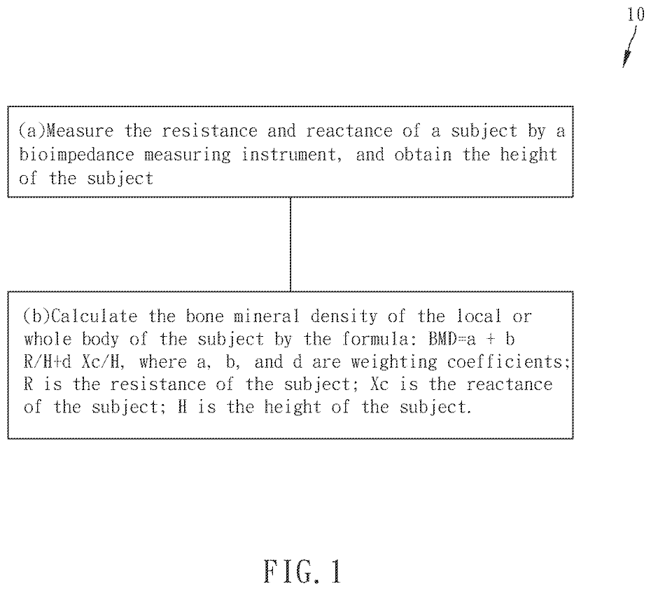

In order to explain the technical features of the present invention in detail, the following is a preferred embodiment, and the descriptions are as follows in conjunction with FIGS. 1-3. The method of bioimpedance technology to evaluate local or whole body bone mineral density 10 provided by the present invention comprises the steps of (FIG. 1):

Step (a) Use a bioimpedance measuring instrument 20 to measure the resistance and reactance of a subject, and obtain the height of the subject.

In this preferred embodiment, the height of the subject is measured by a height rod and manually inputted into the bioimpedance measuring instrument 20, and the accuracy of the height rod is set to 0.5 cm (but not limited to this). Thereby, the height that best meets the current situation of the subject can be obtained, so as to improve the accuracy of the evaluation result of the present invention. In other preferred embodiments, the subject's height can also be directly provided by the subject, and transmitted to the bioimpedance measuring instrument 20 in an automatic and wired/wireless manner. Therefore, the selection of the bioimpedance measuring instrument 20 and the way the bioimpedance measuring instrument 20 obtains the height of the subject are not limited to the present preferred embodiment.

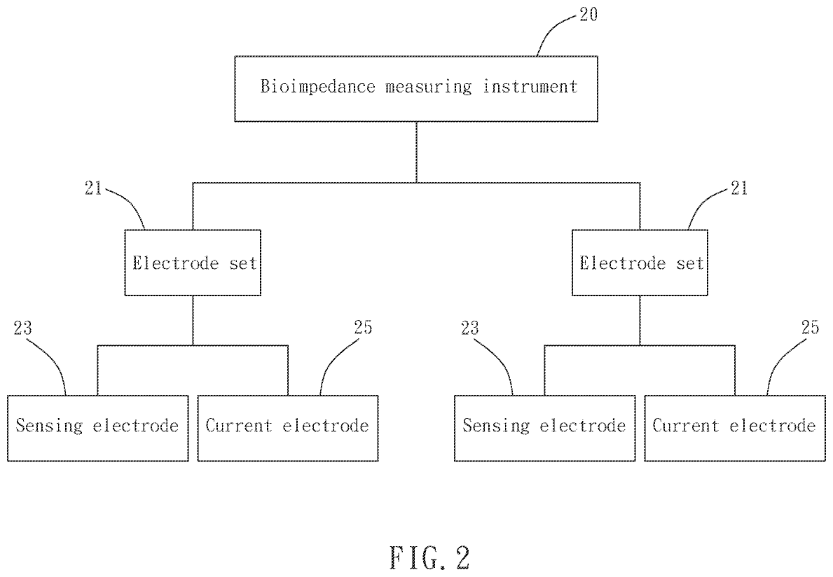

As shown in FIG. 2, in the preferred embodiment, the bioimpedance measuring instrument 20 measures the resistance and reactance of the subject, and the subject performs the measurement of the bioimpedance measuring instrument 20 in a supine position, thereby reducing evaluation error. For the plural electrode sets 21 of the bioimpedance measuring instrument 20, the number of the plural electrode sets 21 is two as an example. The plural electrode sets 21 contact the subject's right hand and right foot to form a measurement loop. Each electrode set 21 comprises a sensing electrode 23 and a current electrode 25. The sensing electrode 23 and the current electrode 25 are separated by 5 cm, based on the characteristics of bioimpedance measurement, thereby improving the accuracy of the evaluation of the present invention. In other preferred embodiments, the subject can also perform the measurement of the bioimpedance measuring instrument 20 in a standing position. The plural electrode sets 21 can also be measured by contacting the subject's hands, feet, right hand to left foot, left hand to right foot, left hand to left foot, etc. Under different measurement modes, the number of the plural electrode sets 21 can also exceed two. The sensing electrode 23 and the current electrode 25 can also be between 1-5 cm or 5-10 cm. Therefore, the number of the plural electrode sets 21 and the manner in which the subject conducts the resistance and reactance measured by the bioimpedance measuring instrument 20 are not limited to this preferred embodiment.

In the present preferred embodiment, the bioimpedance measuring instrument 20 measures the body mineral density of the subject's body (BMDₜₒₜₐₗ), lumbar spine (BMD_{LS}) and right upper limb (BMD_{right arm}), a total of three parts of bone mineral density. In other preferred embodiments, it is also possible to choose to measure other parts of the subject according to needs, and to increase or decrease the measured parts of the subject according to needs.

Step (b) The bone mineral density of the subject's local or whole body is calculated by the following formula:

BMD=a+b R/H+d Xc/H, where a, b, and d are weighting coefficients; R is the resistance of the subject; Xc is the reactance of the subject; H is the height of the subject.

In the present preferred embodiment, the weighting coefficients of a, b, and d are obtained by using DXA for the whole body, lumbar spine, and right upper limb of hundreds of testers, after obtaining the average bone mineral density of the three positions. In other preferred embodiments, the weighting coefficients of a, b, and d can also be obtained after measurement by a quantitative ultrasonic inspection instrument, quantitative computed tomography, or traditional X-ray inspection. And, other parts of the tester can be selected to be measured according to the needs, and the tester's measurement parts can be increased or decreased according to the needs. Therefore, the instrument and the body parts of the tester are not limited to the present preferred embodiment.

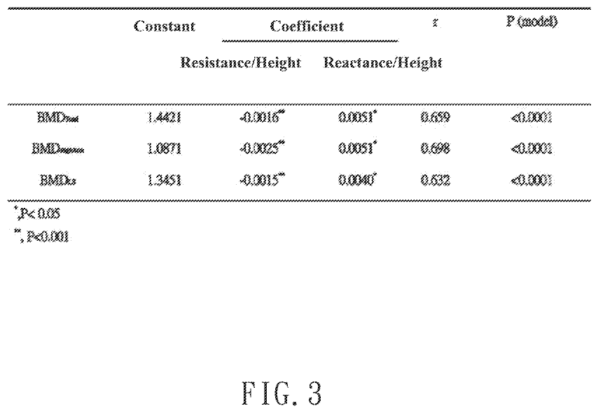

As shown in FIG. 3, it is the result of the present preferred embodiment. In the GLM regression analysis, the corresponding weighting coefficients and correlations show that the R/H and Xc/H values of the subject are positively correlated with the values of the whole body, lumbar spine and right upper limb measured by DXA.

It can be proved that the method of bioimpedance technology to evaluate local or whole body bone mineral density provided by the present invention only uses the bioimpedance measuring instrument to obtain the resistance, reactance, and height of the subject, and the bone mineral density can be measured after calculating with the formula BMD=a+b R/H+d Xc/ii. Compared with the prior art, the present invention is low in cost, fast, and convenient in use.

What is claimed is:

1. A method of bioimpedance technology to evaluate local or whole body bone mineral density, comprising the steps of:
(a) measuring a resistance and a reactance of a subject by a bioimpedance measuring instrument, and obtaining a height of said subject;
(b) calculating the bone mineral density of the local or whole body of said subject by the following formula:
BMD=a+b R/H+d Xc/H, where a, b, and d are weighting coefficients; R is the resistance of said subject; Xc is the reactance of said subject; H is the height of said subject.

2. The method of bioimpedance technology to evaluate local or whole body bone mineral density as claimed in claim 1, wherein in step (a), the method for said bioimpedance measuring instrument to measure the resistance and reactance of said subject is to measure plural electrode sets of said bioimpedance measuring instrument, said plural electrode sets contacting the hands and feet, hands or feet of said subject to form a measurement loop, each said electrode set comprising a sensing electrode and a current electrode, said sensing electrode and said current electrode being 1-10 cm apart.

3. The method of bioimpedance technology to evaluate local or whole body bone mineral density as claimed in claim 1, wherein in step (a), said subject is to perform the measurement of said bioimpedance measuring instrument in a supine, standing or sitting position selectively.
